(19) United States
(12) Patent Application Publication
(10) Pub. No.: US 2014/0377246 A1
(43) Pub. Date: Dec. 25, 2014

(54) ENZYME REPLACEMENT THERAPY FOR TREATING MPS VII RELATED BONE LESIONS USING A CHEMICALLY MODIFIED ENZYME

(71) Applicant: Carol Ann Foundation and International Morquio Organization, Tucson, AZ (US)

(72) Inventors: Shunji Tomatsu, Wilmington, DE (US); Jeffrey Grubb, Petaluma, CA (US); Daniel Rowan, Wauwatosa, WI (US); William Sly, Saint Louis, MO (US)

(73) Assignee: Carol Ann Foundation and International Morquio Organization, Tucson, AZ (US)

(21) Appl. No.: 14/302,016

(22) Filed: Jun. 11, 2014

Related U.S. Application Data
(60) Provisional application No. 61/837,141, filed on Jun. 19, 2013.

(51) Int. Cl. A61K 38/47

(52) U.S. Cl. CPC A61K 38/47; C12Y 302/01031
USPC 424/94.61; 424/94.6

(57) ABSTRACT

The invention relates to a method of treating mucopolysaccharidoses using enzyme replacement therapy with chemically modified lysosomal enzymes. More specifically the method relates to administering chemically modified lysosomal enzymes intraperitoneal injection. In addition, the invention relates to treating type VII mucopolysaccharidoses or mucopolysaccharidoses type VII related bone lesions with a chemical modified β-glucuronidase, wherein the modified β-glucuronidase may be administered 5 weeks after birth, and or may be administered intraperitoneally.

### CROSS REFERENCE TO RELATED APPLICATIONS

This application claims priority to provisional application 61/837,141, filed Jun. 19, 2013, which is hereby incorporated by reference in its entirety.

### GOVERNMENT FUNDING

This work was supported by National Institutes of Health grant no. GM34182. The government of the United States may have certain rights in this invention.

### FIELD OF THE INVENTION

The invention relates to methods of treating mucopolysaccharidoses using enzyme replacement therapy and chemically modified lysosomal enzymes.

### BACKGROUND

The mucopolysaccharidoses (MPS) are a group of lysosomal storage disorders (LSDs) that result from a deficiency of lysosomal enzymes necessary for the degradation of glycosaminoglycans (GAGs). In mucopolysaccharidosis type VII (MPS VII; Sly syndrome) the GAGs, dermatan sulfate, heparan sulfate, chondroitin 4-sulfate, and chondroitin 6-sulfate, accumulate in lysosomes in the absence of the catabolic enzyme β-glucuronidase (GUS)(1). Around 50 different mutations in the GUS gene have been identified producing a wide range of clinical severity (2). MPS VII is characterized by short stature, dysmorphic features, corneal clouding, hepatomegaly, skeletal abnormalities collectively referred to as dysostosis multiplex, and developmental delay. These clinical manifestations become progressively worse over time if left untreated. MPS VII patients with the most severe phenotype have hydrops fetalis prenatally and often are stillborn or survive only a few months. At the other extreme, patients with attenuated manifestations of MPS VII have survived into the fifth decade of life.

Murine models of MPS VII have characteristics similar to the human disease (3,4). MPS VII mice show GAG storage in lysosomes of visceral organs, skeleton, and brain. They have facial dysmorphism, growth retardation, deafness, behavioral deficits, and a shortened lifespan. Radiographic analysis showed significant bone dysplasia including shortened and thick long bones, sclerosis of the calvarium, and a narrow thorax. Microscopically, the epiphyseal growth plate is hypercellular and irregular and osteoblasts in the bone marrow contain vacuoles. In addition, synovial proliferation, vacuolated synovial cells, and articular-synovial synechiae have been described.

Several LSDs have been treated with enzyme replacement therapies (ERTs), which rely on mannose 6-phosphate receptor (M6PR) or mannose receptor-mediated uptake of enzymes into target cells (5-8). This receptor-mediated ERT strategy has been used with substantial success to treat storage in visceral organs in murine MPS VII. However, GAG storage in the central nervous system (CNS) has been resistant to clearance by ERT using conventional doses of enzyme unless begun during the newborn period (9, 10). In several disease models partial correction in some areas of the brain followed repeated injections of large doses of enzyme (11-14). Grubb et al. reported that a chemically modified form of GUS referred to herein as PerT modified β-glucuronidase or PerT-GUS, which was more resistant to clearance from the blood by mannose and mannose 6-phosphate receptors, and showed prolonged circulation (half-life over 18 hours) and was more effective than native enzyme at clearing storage from cortical and hippocampal neurons. Higher levels of enzyme in other tissues suggested improved delivery to other organs as well (15). The mechanism, by which PerT-GUS enzyme escapes uptake by the mannose and mannose 6-phosphate receptors, relies on chemical inactivation of its terminal sugars by treatment of sodium metaperiodate followed by borohydride reduction. How long-circulating PerT-GUS gains entry to some cell types remains unknown. This chemically modified form of GUS (PerT-GUS), may escaped clearance by mannose 6-phosphate and mannose receptors, and reduce CNS storage more effectively than native GUS. However clearance of GAG storage material in bone is limited by the avascularity of the growth plate. In this disclosure, the Inventors compared the skeletal response of MPS VII mice to treatment with 12 weeks of either PerT-GUS or native GUS ERT where treatment began 5 weeks after birth. The Inventors also assessed the skeletal effects of long-term treatment of MPS VII mouse models with PerT-GUS ERT. Micro-CT, radiographs, and quantitative histopathology were used in parallel to define the bone pathology in MPS VII mice and their response to treatments. In addition, some subjects after receiving repeated intravenous injections or infusions suffer from collapsed veins and have difficulty with continued intravenous treatment. The Inventors have addressed this problem by disclosing a method of ERT which uses intraperitoneal administration.

### SUMMARY OF THE INVENTION

A method of treating mucopolysaccharidoses by using enzyme replacement therapy with chemically modified lysosomal enzymes by intraperitoneal injection. A method of treating type VII mucopolysaccharidoses by administering a chemically modified β-glucuronidase by intraperitoneal injection. A method of treating type VII mucopolysaccharidoses using enzyme replacement therapy by administering a chemically modified β-glucuronidase, beginning at 5 weeks after birth. A method of treating a type VII mucopolysaccharidoses related bone lesion using enzyme replacement therapy beginning about 5 weeks after birth wherein bone mineral density improves.

### DESCRIPTION OF THE FIGURES

FIG. 1 shows histopathology of the knee joint of 17 week-old intravenous (IV) GUS and PerT-GUS treated MPS VII mice. Images are of the growth plate and articular cartilage. Tissue was stained using toluidine blue.

FIG. 2 shows quantitative analysis of histopathology of 17 week-old IV GUS (n=4) and PerT-GUS (n=4) treated mice. (A) Articular cartilage and growth plate thickness. (B) Articular cartilage and growth plate cellularity (the number of chondrocytes in a given area of the articular cartilage or growth plate). (C) Average cell area of chondrocytes in the proliferative zone of the growth plate and articular cartilage. (D) Mean number of cells per column in the proliferative zone of the growth plate in GUS and PerT-GUS treated mice. (E) The perimeter to length ratio of the growth plate in GUS and PerT-GUS treated mice, this is a measure of the irregularity of the growth plate. * represents p<0.05. AC: articular cartilage, GP: growth plate

FIG. 3 shows micro-CT reconstructions of the knee joints of 17 week-old IV GUS and PerT-GUS treated MPS VII mice. The left side of each picture shows the unsectioned bones of the knee joint, the right side of each picture shows a sagittal cross-section through the midline of the knee joint.

FIG. 4 shows micro-CT reconstructions of the cervical spine of 17 week-old IV GUS and PerT-GUS treated MPS VII mice. Each picture shows unsectioned bone (left) and a midline sagittal cross section through the cervical spine (right).

FIG. 5 shows radiographs of the legs (A) and spine (B) of 17 week-old IV GUS and PerT-GUS treated mice.

FIG. 6 shows three dimensional micro-CT reconstructions of knee joints of wild-type, untreated MPS VII, and intraperitoneal (IP) PerT-GUS treated MPS VII mice. Each picture shows unsectioned bone (left side) or sagittal-sectioned bone (right side). Cross sections are sagittal through the midline of the knee joint. The long arrows identify areas of thickened cortical bone. The short arrows identify abnormal exophytic bone formations on articular surfaces. Ages of wild-type and untreated MPS VII mice are 5, 23, and 36 weeks old. Ages of mice treated with 2 mg/kg PerT-GUS were 27, 41, and 57 weeks-old.

FIG. 7 shows three dimensional micro-CT reconstructions of the cervical spine of wild-type, untreated MPS VII, and IP PerT-GUS treated MPS VII mice. Each picture shows unsectioned bone (left side) or sagittal-sectioned bone (right side). Cross sections are sagittal through the midline of the spine. The arrow on the 5-week-old MPS VII spine identifies an area of decreased bone formation. Arrows on 23- and 36-week-old MPS VII mice identify areas of extra bone formation. Wild-type and MPS VII mice ages are 5, 23, and 36 weeks old. The mouse age treated with 2 mg/kg PerT-GUS is 38 weeks old.

FIG. 8 shows radiographs of wild-type, untreated MPS VII, and IP PerT-GUS treated MPS VII mice. Radiographs of legs, spine, and ribcages. Arrows identify areas of bone thickening and increased radiodensity in the femur, cervical spine, and ribs. Leg measurements in X-ray pictures. Values are means (wild-type, n=5; MPS VII, n=4; PerT-GUS treated MPS VII, n=3) with error bars representing one standard deviation. *significantly decreased compared with wild-type **significantly decreased compared with untreated MPS VII mice. See FIG. 12 for measurement details.

FIG. 9 shows histopathology of wild-type (36 weeks old), untreated MPS VII (32 weeks old), and IP PerT-GUS treated MPS VII mice (27 weeks old). Images are of the growth plate, articular cartilage, trabecular bone/bone marrow, and cortical bone. Tissue was stained with toluidine blue. Arrows on growth plate and articular cartilage micrographs identify distended chondrocytes. Arrows on cortical bone micrographs identify distended osteocytes, which are more prevalent in untreated MPS VII bone than in PerT-GUS treated MPS VII bone. GP: growth plate, BM: bone marrow, M: meniscus, AC: articular cartilage.

FIG. 10 shows quantitative analysis of histopathology of tibias in wild-type (n=5), untreated MPS VII (n=4), and IP PerT-GUS treated MPS VII mice (n=5). (A) Shows growth plate and articular cartilage thickness. (B) Number of chondrocytes in a given area of the articular cartilage (cellularity). (C) Number of chondrocytes in a given area of the proliferative zone of the growth plate (cellularity). (D) Cross sectional cell area of chondrocytes in the proliferative zone of the growth plate or articular cartilage, values reported are means of measurements taken for all chondrocytes in one 40× microscope field. (E) Number of cells in each proliferative zone column in the growth plate. Values reported are means of the number of cells in each column in one 40× microscope field. (F) Perimeter to length ratio measured as shown in FIG. 14. * represents p<0.05. Error bars represent one standard deviation.

FIG. 11 shows leg measurements. Lines show the locations at which tibia and foot lengths and femur thickness are measured.

FIG. 12 shows cell area in growth plate region. Contoured lines are traced around the cells and area within the line is measured and calculated as cell area.

FIG. 13 shows perimeter to length ratio in growth plate region. Perimeter (contoured line) and length (straight line) are measured as shown by the black lines.

FIG. 14 shows histopathology in articular cartilage and growth plate region of wild-type and untreated MPS VII mice (left panel, newborns; middle panel, 2.5 weeks old; right panel, 5 weeks old). Images are of the growth plate and articular cartilage. Tissue was stained with toluidine blue. Arrows on growth plate and articular cartilage micrographs identify distended chondrocytes.

### DETAILED DESCRIPTION OF THE INVENTION

Mucopolysaccharidosis (MPS) type VII is a lysosomal storage disease caused by deficiency of the lysosomal enzyme β-glucuronidase (GUS), which is involved in the degradation of glycosaminoglycans (GAGs). In order to increase the time of exposure to high levels of β-glucuronidase (GUS) in the blood and tissues, β-glucuronidase was chemically modified by sodium metaperiodate followed by sodium borohydride reduction (PerT GUS) to prevent uptake by the M6P or mannose receptor pathway. While not wishing to be bound by theory, it is thought that this chemical modification results in removal or modification of mannose and mannose-6-phosphate exposed sugars on GUS. The result is that PerT GUS is not bound and removed from the blood by cell surface mannose and mannose-6-phosphate receptors located on the luminal surface of the blood vessels. Unmodified native GUS was cleared with a t_{1/2}of 11.7 min by the MR and the M6PR clearance systems while the clearance of modified β-glucuronidase (PerT-GUS) was dramatically prolonged to a t_{1/2}of 18.5 h. PerT-GUS ERT therapy was also able to clear GAG storage material from the CNS in a mouse model of MPS VII. (15) However, because clearance of GAG storage material from bone is thought to be limited by the avascularity of the growth plate, the effectiveness of PerT-GUS on the treatment of bone lesions remains unknown. In addition, it is sometimes difficult for clinicians to find veins suitable for intravenous injection in subjects receiving repeated intravenous injections or infusions. Particularly when the subject is of small size. The term “subject” as used herein is meant to refer to mammalian subjects including experiential animals, and human subjects, including newborn human subjects. Intraperitoneal administration of enzyme replacement therapy was not considered feasible because absorption of enzymes from the intraperitoneal cavity was either unknown or thought to be unlikely. This was confirmed by the Inventors (unpublished results) The Inventors disclose a method of ERT administration that utilizes a PerT modified enzyme and intraperitoneal injection. In the particular example disclose below, a PerT modified GUS is administered intraperitoneally in a mouse model of MPS VII.

Treatment of Bone Lesions: Short Term Protocol

A limitation of ERT for LSDs has been the inability to correct bone pathology because of the avascularity of the growth plate. It was also thought that unless ERT treatment began in the neonatal period it will not be effective (9, 16, 24). In previous studies, the response to treatment of MPS VII from birth with intravenous native GUS enzyme was shown to improve growth, fertility, longevity, and histology of visceral organs. However, the response of bone (chondrocytes) to ERT was limited even if treatment began at birth (9,11,16). Given the greatly prolonged blood clearance of PerT-GUS, the inventors reasoned that PerT-GUS may be effective when administered outside of this neonatal window. To this end the Inventors designed a short term treatment protocol whereby a MPS VII mouse model was treated with intravenous injections of either native GUS or Pert-GUS weekly, at 2 mg per kilogram of the subject to be treated, for 12 weeks, starting 5 weeks after birth. In this short term protocol, several quantitative measurements of histopathology showed significant improvement in mice treated with PerT-GUS compared with native GUS treatment. These results indicate that when using Pert-GUS, treatment need not start at birth to provide beneficial therapeutic effects.

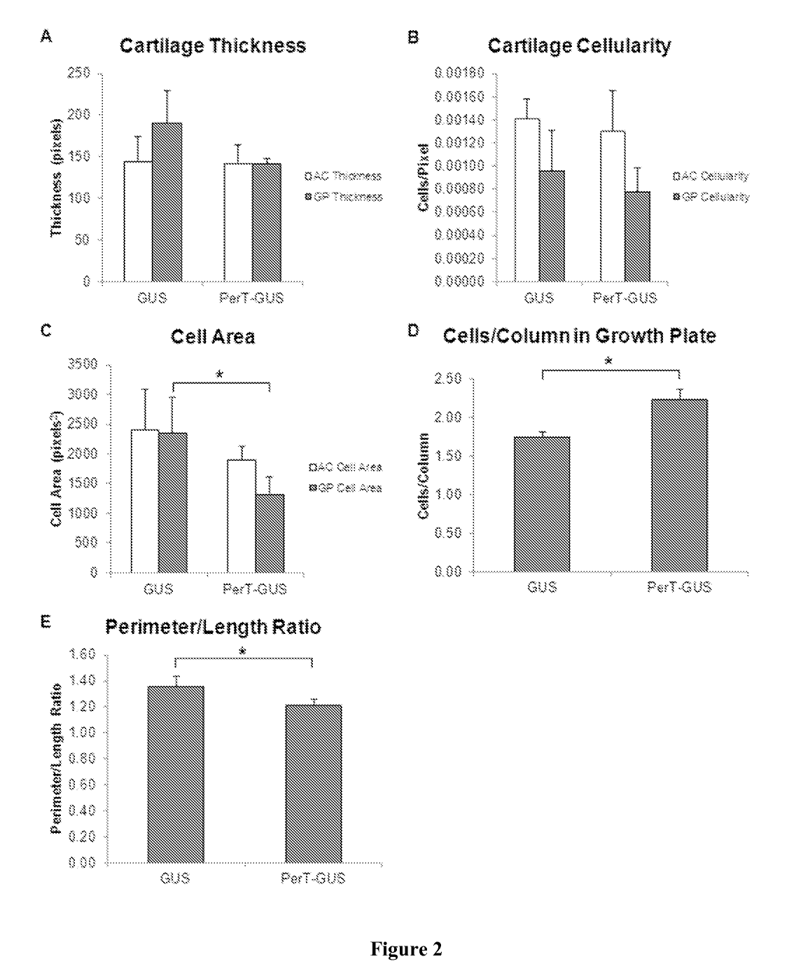

The direct comparison of native GUS and PerT-GUS confirmed that PerT-GUS treated mice have significantly reduced storage material at the growth plate and while not statistically significant, storage material was also reduced in the articular cartilage, as indicated by cell area measurements (FIG. 2C). In addition, the Inventors show that the growth plate is less disorganized in PerT-GUS treated mice, compared with GUS treated mice as indicated by an increase in number of cells per growth plate column (FIG. 2D) and a perimeter/length ratio (FIG. 2E) which is significantly reduced towards normal in PerT-GUS treated mice.

Micro-CT studies showed greater reduction in bone mineral density (BMD) with PerT-GUS treatment. These findings were supported by the X-ray findings of lower radiodensity in PerT-GUS treated mouse legs as well as reduced femur thickness compared to those of GUS treated mice. Histopathological analysis also showed reduced storage material and a more organized growth plate in PerT-GUS treated mice compared with GUS treated mice.

Intraperitoneal Treatment

The Inventors made the surprising discovery that PerT modification of GUS enabled the enzyme to be suitable for intraperitoneal administration. The Inventors observed that the subjects' response to multiple intraperitoneal injections of PerT-GUS were equivalent to those receiving multiple intravenous injections. Despite the fact that PerT-GUS was taken up poorly by peritoneal lining cells, intraperitoneally infused enzyme reached the same concentrations in the blood as intravenously infused PerT-GUS, after a 30-60 minute delay (data not shown). This contrasts with earlier observations that the native enzyme was much less effective if administered IP because much of the delivered dose was taken up by peritoneal lining cells and never reached the circulation. While not wishing to be bound by theory, it is thought that because PerT-GUS is not taken up by cells of the peritoneal lining it may enter the subject's circulation by way of the lymphatic system. The Inventors believe that PerT modification is responsible for providing these new properties to GUS, and that PerT modification of other lysosomal enzymes would provide the same or similar properties to those enzymes, including prolonged half-life in circulation and enabled delivered by intraperitoneal administration. It is envisioned that subjects suffering from type VII mucopolysaccharidoses may receive intraperitoneal administration of PerT-GUS during a course of enzyme replacement therapy. Similarly, it is envisioned that subjects suffering from other LSDs may receive intraperitoneal administration of a PerT modified lysosomal enzyme during a course of enzyme replacement therapy. Non-limiting examples of LSDs and their respective deficient lysosomal enzyme which are expected to be provided with prolonged half-life in circulation and enabled delivered by intraperitoneal administration after PerT modification include: Morquio syndrome, deficient in N-acetylgalactosamine-6-sulfatase; Hurler syndrome, deficient in Iduronidase; Hunter syndrome, deficient in Iduronate-2-sulfatase; Sanfilippo syndrome, deficient in Alpha-N-acetylglucosaminidase; Gaucher's disease, deficient in beta-glucosidase; Fabry disease, deficient in alpha-galactosidase; Hurler syndrome, deficient in α-L-iduronidase; Maroteaux-Lamy syndrome, deficient in N-acetylgalactosamine 4-sulfatase; and Pompe disease deficient in acid alpha-glucosidase. A preferred example is type VII mucopolysaccharidoses, deficient in β-glucuronidase, as exemplified in this disclosure.

The use of intraperitoneal administration to deliver ERT will be a benefit to subjects who receive repeated administrations of enzymes, in particular those which are of a small size. It is anticipated that most subjects will begin ERT therapy early in life and these subjects will include neonatal subjects. It is also envisioned that IP administration may be accomplished with the aid of various pumps and/or peritoneal infusion or injections ports, well known in other methods of treatment, by way of example the delivery of chemotherapy to cancer patients. Intraperitoneal delivery of ERT by way of a peritoneal port would allow repeated access to the intraperitoneal cavity by the clinician with minimum trauma to the subject.

Treatment of Bone Lesions: Long Term Protocol

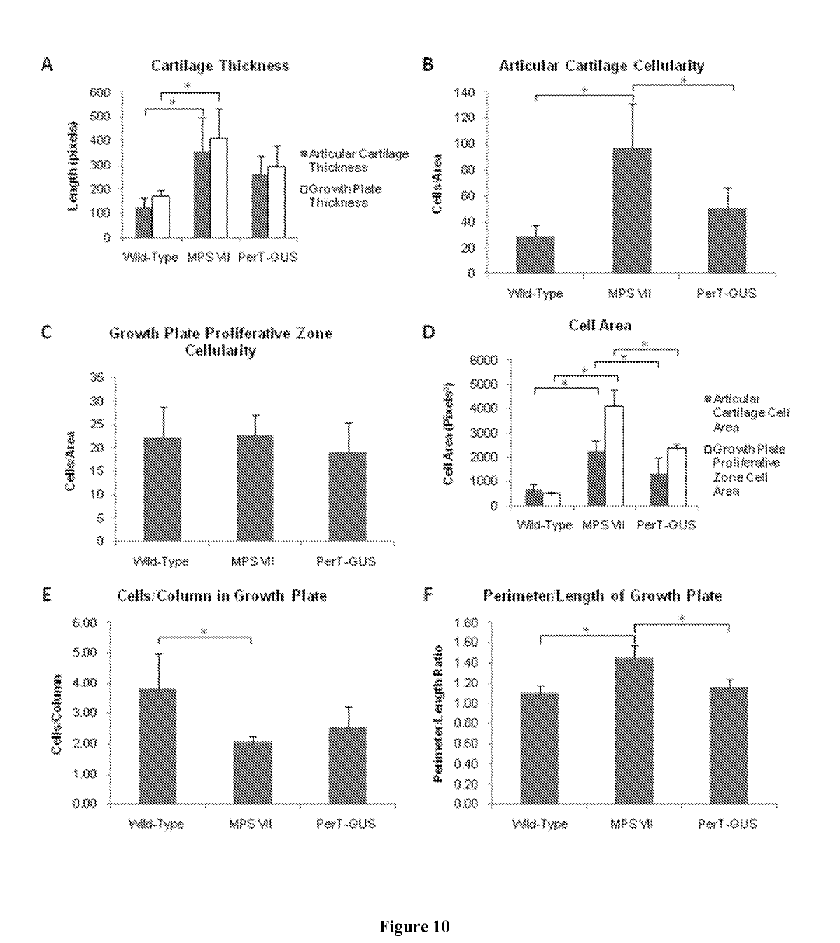

It was unknown whether long term PerT-GUS treatment would be effective enough to reduce bone lesions due to MPS VII, to a state comparable to a non-disease subject. To that end the Inventors designed a treatment protocol whereby a MPS VII mouse model was treated with PerT-GUS at 2 mg per kilogram of the subject to be treated, weekly from birth until 6 weeks, then with PerT-GUS at 2 mg per kilogram of the subject to be treated, administered every other week until 57 weeks of age. Based on the observation described above, all treatments were administered by intraperitoneal injection. Effectiveness was evaluated using micro-CT, X-rays, and histopathology. After 57 weeks, The quantitative histological analysis showed that long-term IP injected PerT-GUS ERT improves epiphyseal growth plate organization and GAG storage and reduces growth plate thickness, cell size of chondrocytes, perimeter/length ratio of growth plate, and abnormal proliferation of articular and meniscal cartilage and connective tissue in knee joints. This study showed significant reduction in size of chondrocytes (both articular and epiphyseal chondrocytes, which were half of the size of untreated chondrocytes: FIG. 10D).

long term PerT-GUS ERT therapy showed the correction of skeletal pathology in a mouse model of MPS VII. In MPS VII mice treated with PerT-GUS ERT. Micro-CT and X-rays demonstrated marked radiographical improvements in bone lesions of legs, ribs, and spine. Histopathology also showed substantial improvements in skeletal GAG storage and morphology. In the long term protocol, micro-CT and radiographs analysis demonstrated that MPS VII mice treated with IP injected PerT-GUS from birth had substantial correction of bone pathology. The Inventors show that PerT-GUS treatment from birth to more than 6 months of age reduced cortical bone thickening and reduced the amount of shortening seen in long bones of the leg. In addition, PerT-GUS reduced exophytic bone formation, diminished spinal stenosis, and normalized radiodensity of the cervical spine and ribs in the MPS VII mice. The BMD of PerT-GUS treated MPS VII mice was reduced to the level of wild-type mice. Thus, IP injected PerT-GUS treatment addresses major components of the dysostosis multiplex associated with MPS VII. The Inventors have shown that long-term PerT-GUS treatment prevents skeletal pathology to an extent that would impact the quality of life of a human subject Similar results in humans may reduce the need for corrective surgeries and improve the quality of life in MPS VII patients.

PerT-GUS

PerT-GUS was prepared as previously described in U.S. application Ser. No. 12/042,601, published as U.S. Published Application No. US 2009/0041741 A1, and incorporated herein by reference in its entirety. In summary, isolated GUS was treated with periodate and borohydride without significantly reducing the enzymatic activity or stability. It is expected that these same methodology may be applied to other lysosomal enzymes to produce other PerT modified lysosomal enzymes.

Generation of Stable Cell Lines Secreting GUS

Using DNA cloning techniques, the cDNA sequence encoding the full length cDNA for human -glucuronidase (GUS) (Genbank Accession # NM 000181)(SEQ ID NO:1) was sub cloned into the mammalian expression vector pCXN (32) The plasmid was introduced into the Chinese hamster ovary cell line, CHO-KI (33) by electroporation (34). After selection in growth medium, high level expressing clones were identified by measuring GUS activity secreted into the conditioned medium. The highest-producing clone was scaled up and secreted enzyme was collected in protein-free collection medium PF-CHO. Conditioned medium collected in this way was pooled, centrifuged at 5000×g for 20 min and the supernatant was collected and frozen at 20° F. GUS was then isolated using conventional column chromatography or antibody affinity techniques.

Treatment of Purified GUS with Periodate and Borohydride: PerT Modification

In order to inactivate the mannose and mannose 6-phosphate recognition sites on GUS, the enzyme was treated by a well-established procedure utilizing reaction with sodium meta-periodate followed by sodium borohydride (35, 36). Approximately 10 mg of purified GUS was treated with a final concentration of 20 mM sodium meta-periodate in 20 mM sodium phosphate, 100 mM NaCl pH 6.0 for 6.5 h on ice in the dark. The reaction was quenched by the addition of 200 mM final concentration ethylene glycol and incubated for an additional 15 min on ice in the dark. Afterwards, this mixture was dialyzed against 2 changes of 20 mM sodium phosphate, 100 mM NaCl pH 6.0 at 4° C. The periodate treated, dialyzed enzyme was then treated with the addition of 100 mM final concentration sodium borohydride overnight on ice in the dark to reduce reactive aldehyde groups. After this treatment, the enzyme was dialyzed against two changes of 20 mM sodium phosphate, 100 mM NaCl, pH 7.5 at 4° C. The final dialyzed enzyme was stored in this buffer at 4° C. where it was stable indefinitely.

Treatment Amounts and Treatment Periods

The above treatment regimens and dosages of PerT GUS administered by intravenous or intraperitoneal infusion are non-limiting. A skilled artisan may determine the treatment dosages based on a particular subject and the severalty of the GUS deficiency or the severity of bone lesions being treated. It is anticipated that treatment regimens of PerT-GUS will vary. Treatment amounts may be from about 0.1 mg/kg to about 1 mg/kg, about 1 mg/kg to about 2 mg/kg, about 2 mg/kg to about 3 mg/kg, about 3 mg/kg to about 4 mg/kg, about 4 mg/kg to about 6 mg/kg about 6 mg/kg to about 12 mg/kg of the subject being treated. A preferable amount of PerT GUS administered is about 2 mg/kg of the subject being treated and a preferred route of administration is by one or more intraperitoneal injections.

The treatment periods described herein are non-limiting. A skilled artisan may determine the start and duration of the treatment period based on a particular subject and the severalty of the bone lesions being treated. It is anticipated that treatment periods would be between about 12 and about 57 weeks and may start at or after birth. By way of example, a treatment period my start 5 weeks after birth.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### EXAMPLES

### Materials and Methods

MPS VII Tolerant Mouse

A tolerant mouse model for MPS VII (4) was developed from the original Birkenmeir GUS deficient mouse (gusmps/mps) (17) and has been used for evaluation of the effectiveness of a variety of experimental treatments (11,15, 18-20). This mouse has characteristics similar to humans with MPS VII including a shortened face, facial dysmorphism, growth retardation, deafness, shortened lifespan, and behavioral deficits. In addition, it is immunotolerant to administered human GUS.

Purification of GUS

GUS was purified by a multistep procedure with conventional column chromatography as described (15). Purified enzyme was frozen at −80° C. where it was stable indefinitely until thawed for treatment with periodate.

Treatment of GUS with Periodate and Borohydride

The M6P and mannose recognition sites on GUS are contained in the oligosaccharide side chains of the enzyme. To inactivate the exposed carbohydrates, the enzyme was treated with sodium metaperiodate followed by sodium borohydride (15). At the final step, the enzyme was dialyzed against two changes of 20 mM sodium phosphate, 100 mM NaCl (pH 7.5) at 4° C., and was stable stored in this buffer at 4° C. before use.

Short Term Protocol: Comparison of Response to Treatment of MPS VII Mice with Native GUS and PerT-GUS

MPS VII mice were treated intravenously (IV) with 2 mg/kg native GUS (n=4) or PerT-GUS (n=4) for 12 weeks beginning at 5 weeks of age. One week after the last treatment, mice were euthanized and tissues were treated in using the protocol described below for long-term treatment with PerT-GUS (Examples 1-3).

Long-Term Protocol: Treatment of MPS VII Mice with PerT-GUS Intraperitoneally

MPS VII mice were treated Intraperitoneally (IP) with a fixed dose of 30,000 units (7 micrograms) at birth (0-1 day) and at 7 days of age, and with 2 mg/kg weekly on days 14, 21, 28, 35 and 42 with IP infusions of PerT-GUS (2 mg/kg body weight). After 6 weeks of age, mice received 2 mg/kg of the enzyme IP every other week until 27-57 weeks of age. One week after the last infusion, tissues from untreated (n=4) or PerT-GUS treated MPS VII mice (n=5; ages 27, 38, 41, 57, and 57 weeks old) were perfused at necropsy with 25 mM Tris and 140 mM NaCl (pH 7.2), fixed in 2% paraformaldehyde and 4% glutaraldehyde, postfixed in osmium tetroxide, and embedded in Spurr resin. For evaluation of lysosomal storage, toluidine blue-stained 0.5-μm-thick sections of knee joints were assessed by light microscopy. The Inventors also euthanized untreated MPS VII mice at 1 day old and ages 2.5, 5, 10, 23, 29, 32, and 36 weeks old and age-matched wild-type mice to understand the progression of the disease (Examples 4-8).

Micro-CT Analysis and Radiography

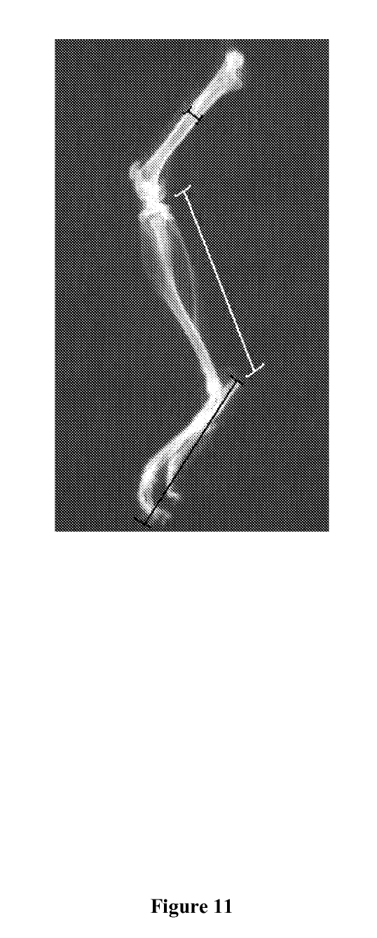

Mice were euthanized using CO₂. At dissection, leg bones, spines, and ribs were placed in 95% ethanol. A micro-CT scan was performed on each bone using a Scanco μCT40 system (Scanco Medical; Brüttisellen, Switzerland) according to manufacturer's instructions (21). Scans were focused on cervical vertebrae 1 and 2 and the knee joint. The bones were then fixed in formalin in preparation for the micro-CT imaging, which was performed on a micro-CT scanner at 16-μm isotropic voxel size, with 250 projections, integration time of 300 msec, photon energy of 50 keV, and current of 160 μA. A three dimensional reconstruction of each bone was made and the bone mineral density (BMD) of each knee joint was measured. Radiographs were also done for each leg, spine, and ribcage and compared. Leg measurements were recorded using plain radiographs (FIG. 11). Measurements were recorded on mice older than 10 weeks, and the mean length measurement and standard deviation were calculated.

Quantitative Analysis of Histopathology

Cartilage thickness: The thickness of the tibia growth plate or articular cartilage was measured at five different places and averaged. This average for each mouse was then used to calculate the mean cartilage thickness for wild-type, untreated MPS VII, GUS treated MPS VII, and PerT-GUS-treated MPS VII groups.

Cellularity: The number of cells in three predetermined areas of equal size in the tibia growth plate proliferative zone and articular cartilage were counted and averaged. The values reported are means and standard deviations of the average cellularity for the mice in each group.

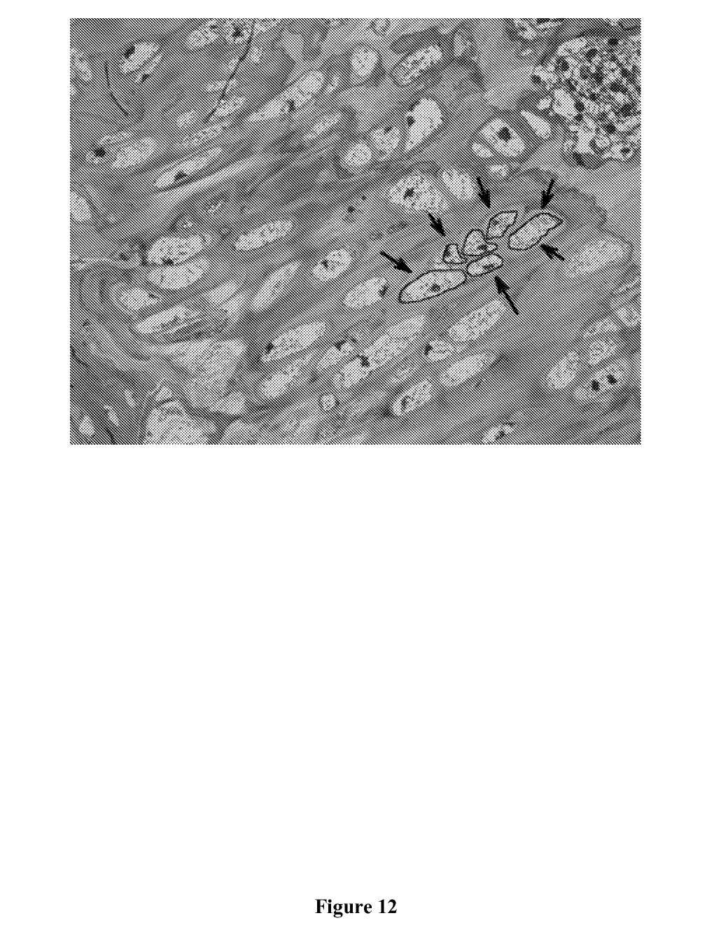

Cell Area: Cells in the proliferative zone of tibia growth plate and articular cartilage were outlined as shown in FIG. 12 and Image J (National Institutes of Health, Bethesda, MD) was used to calculate the area within the outlined area. An average cell area was calculated for the proliferative zone of the growth plate and articular cartilage for each mouse. Areas reported are means with standard deviations of the average area for each mouse group.

Cells/Column: The number of cells stacked in columns perpendicular to the long axis of the tibia growth plate was counted, and the mean value was reported.

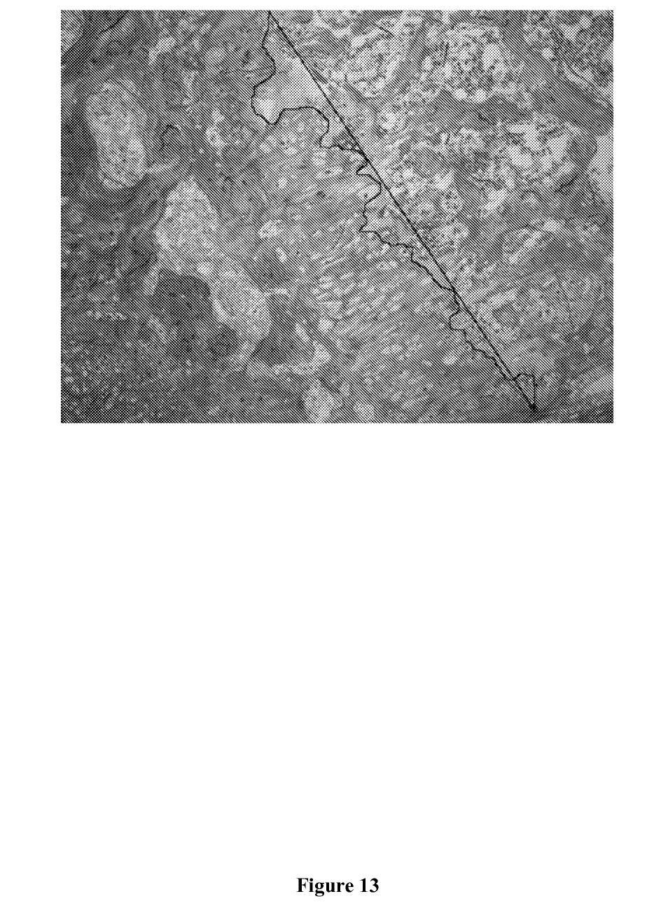

Perimeter/Length Ratio: The length and perimeter of the tibia growth plate region were measured as shown in FIG. 13 (22). The values reported are means and standard deviations for each mouse group.

Short Term Protocol: GUS and PerT-GUS ERT Treatment of MPS VII Mice

Examples 1-3 were preformed to examine the effects of the short term treatment protocol with PerT-GUS.

### Example 1

Growth Plate and Articular Cartilage Histology

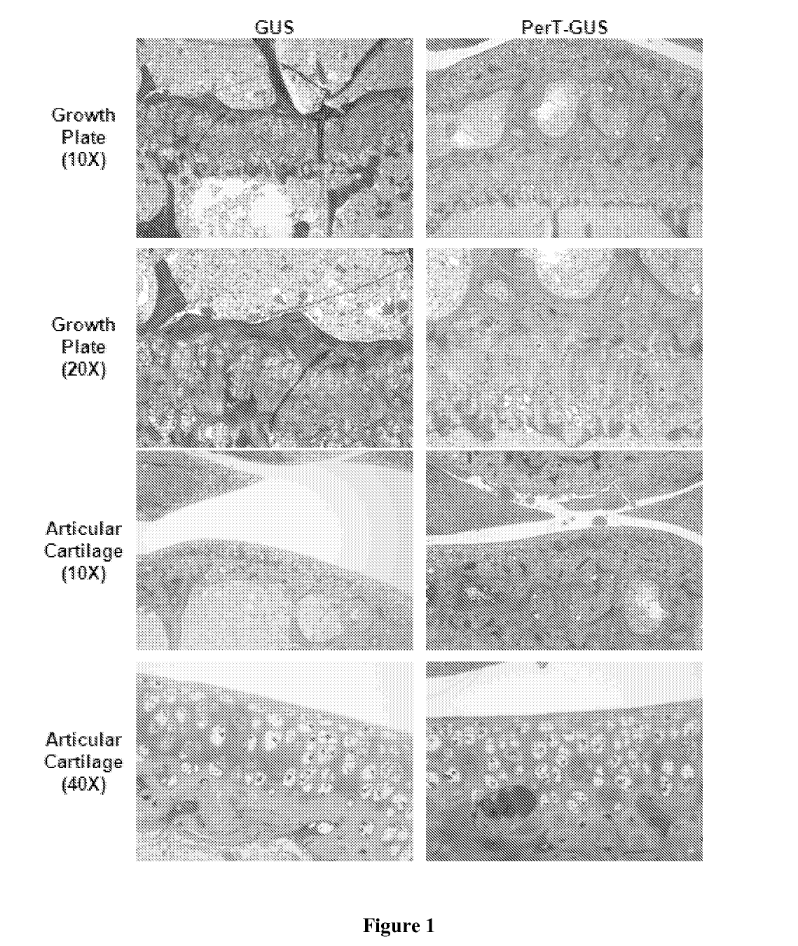

Growth Plate: The resting, proliferative, and hypertrophic zones of the growth plates in GUS and PerT-GUS treated mice contained enlarged and vacuolated cells (FIG. 1). Resting and proliferative zonal chondrocytes appeared larger in size in GUS treated mice compared with PerT-GUS treated mice. The growth plate was thicker and less organized in GUS mice. The normal columnar structure of the proliferative zone was also better preserved in PerT-GUS treated mice compared with GUS treated mice.

Articular Cartilage: Cells of the articular cartilage and meniscus were enlarged and vacuolated in both GUS and PerT-GUS treated mice. The articular cartilage chondrocytes were moderately smaller in PerT-GUS treated mice than in GUS treated mice. Cells in the meniscus of PerT-GUS treated mice contained noticeably less storage material compared with meniscal chondrocytes in GUS treated mice.

### Example 2

Quantitative Histopathological Analysis

To assess the morphology of the growth plate and articular cartilage in GUS and PerT-GUS treated MPS VII mice, the Inventors measured the thickness of the cartilage layer in the growth plate and articular cartilage, the cellularity in the articular cartilage and proliferative zone of the growth plate, a cross-sectional area of chondrocytes in the articular cartilage and proliferative zone of the growth plate as an estimate of cell volume, the mean number of cells aligned in columns perpendicular to the growth plate, and the ratio of the perimeter of the growth plate to its length as an indication of the amount of irregularity in the morphology of the growth plate (FIGS. 12 and 13).

These measurements supported our histological observations. The thickness of the articular cartilage in GUS and PerT-GUS treated mice was similar, however the growth plates in GUS treated MPS VII mice showed a trend towards increased thickness compared with PerT-GUS treated mice (p=0.51; FIG. 2A). The cellularity of the articular cartilage and growth plate was similar in GUS and PerT-GUS treated mice (FIG. 2B). Cross sectional cell area was lower in the proliferative zone of the growth plate (p<0.05) of PerT-GUS treated mice compared with GUS treated mice. Cross sectional cell area in articular cartilage chondrocytes was also lower in PerT-GUS treated mice compared with GUS treated mice, however this difference did not reach statistical significance (FIG. 2C). Two quantitative measures of growth plate organization (cells/column and growth plate perimeter/length ratio) showed that the growth plate of PerT-GUS treated mice is significantly more organized than that of GUS treated mice. The mean number of cells per proliferative zone column was higher in PerT-GUS treated mice (p<0.05; FIG. 2D) and the growth plate perimeter/length ratio was lower in PerT-GUS treated mice compared with GUS treated mice (p<0.05; FIG. 2E).

### Example 3

Micro-CT and Radiographic Findings

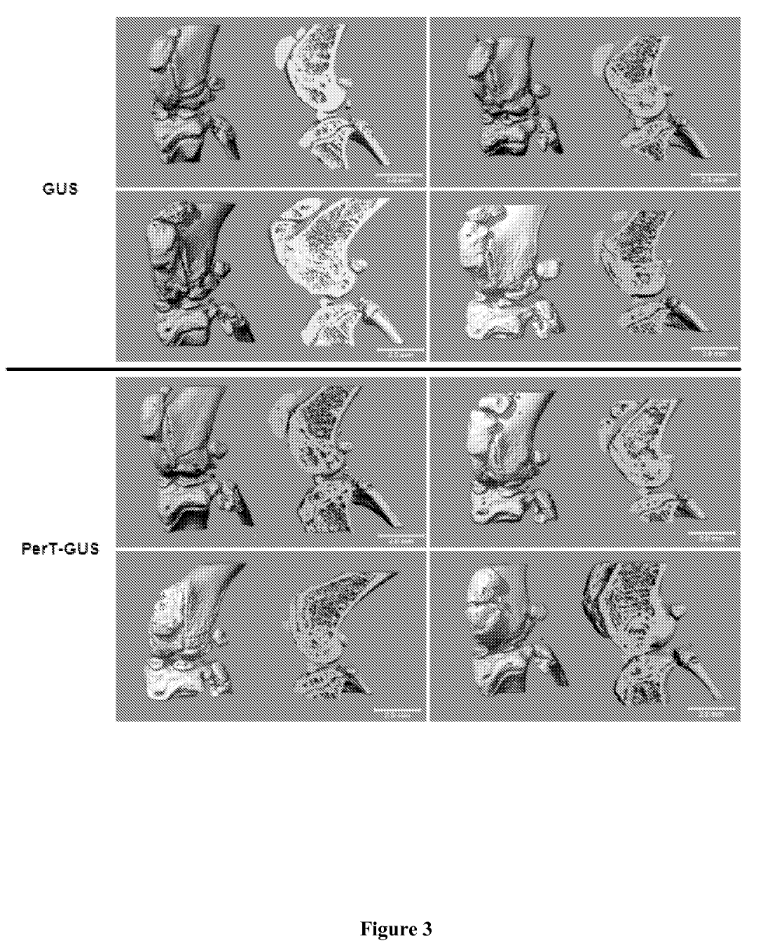

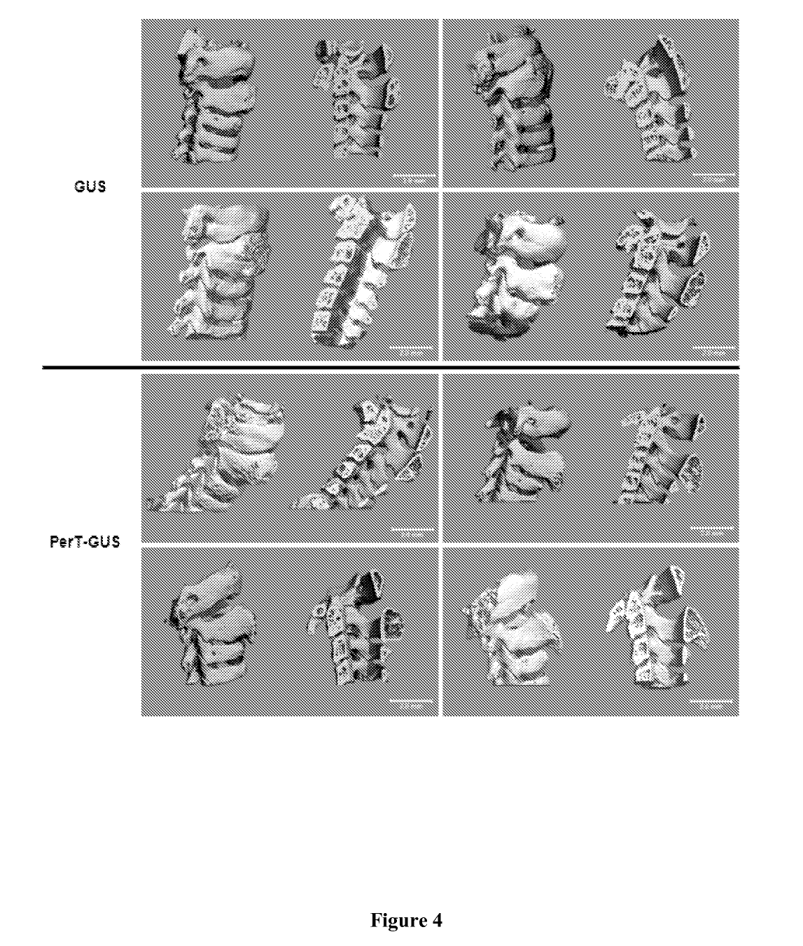

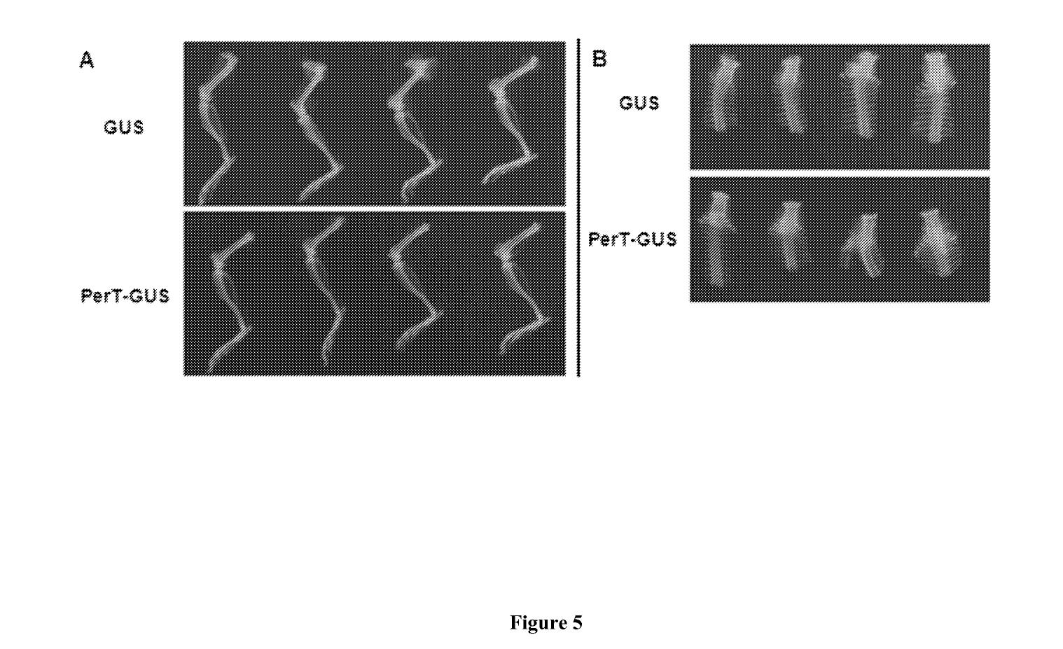

Micro-CT analysis of the bones of the knee joint (FIG. 3) and spine (FIG. 4) of GUS and PerT-GUS treated MPS VII mice showed that both GUS and PerT-GUS treatments significantly reduced the exophytic bone formation and cortical bone thickening which is seen in untreated MPS VII mice. Micro-CT scans also provided the (BMD) of the bones of the knee joint. GUS treated mice had a mean BMD of 459.11±9.59 mgHA/ml, PerT-GUS treated mice had a significantly lower BMD 444.86 mgHA/ml (p<0.05). This reduced BMD is evident in leg X-rays of GUS and PerT-GUS treated MPS VII mice (FIG. 5). Measurement of the thickness of the femur at its midpoint showed that the femurs of GUS treated mice remained abnormally thick (1.25±0.29 mm) compared with the femurs of PerT-GUS treated mice (1.13±0.25 mm; p<0.05). However, tibia length is similar in both GUS (1.60±0.04 cm) and PerT-GUS (1.63±0.03 cm) treated mice.

Long Term Protocol: Effects of Long-Term Treatment with PerT-GUS

Examples 4-8 were preformed to examine the effects of the long term treatment protocol with PerT-GUS.

### Example 4

Micro-CT Findings

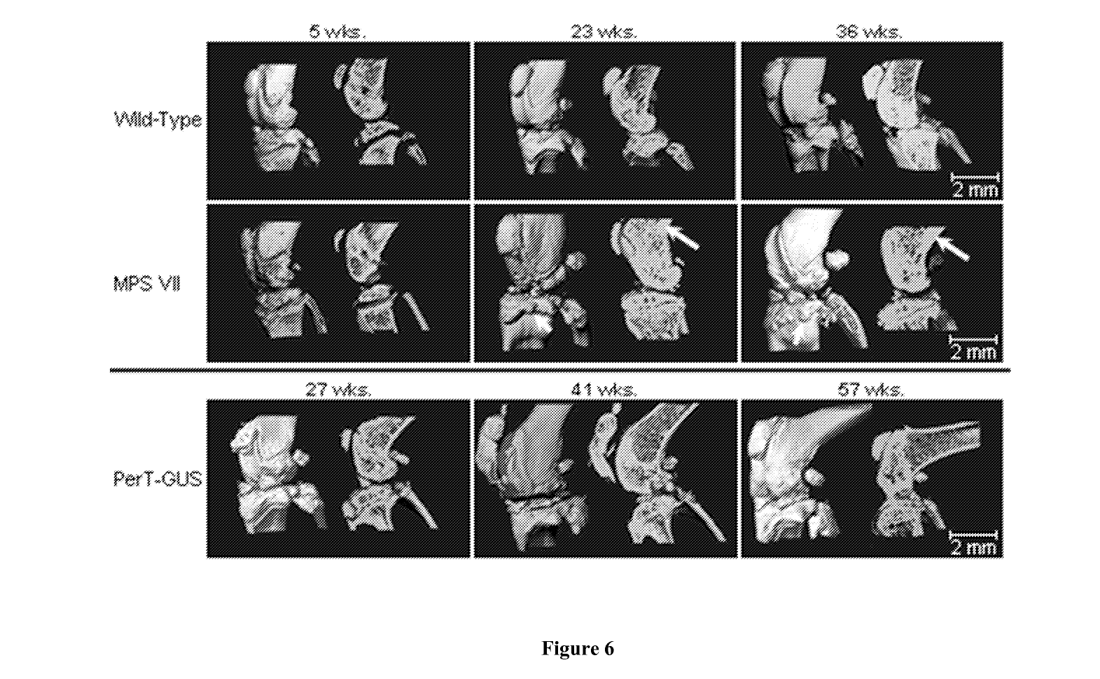

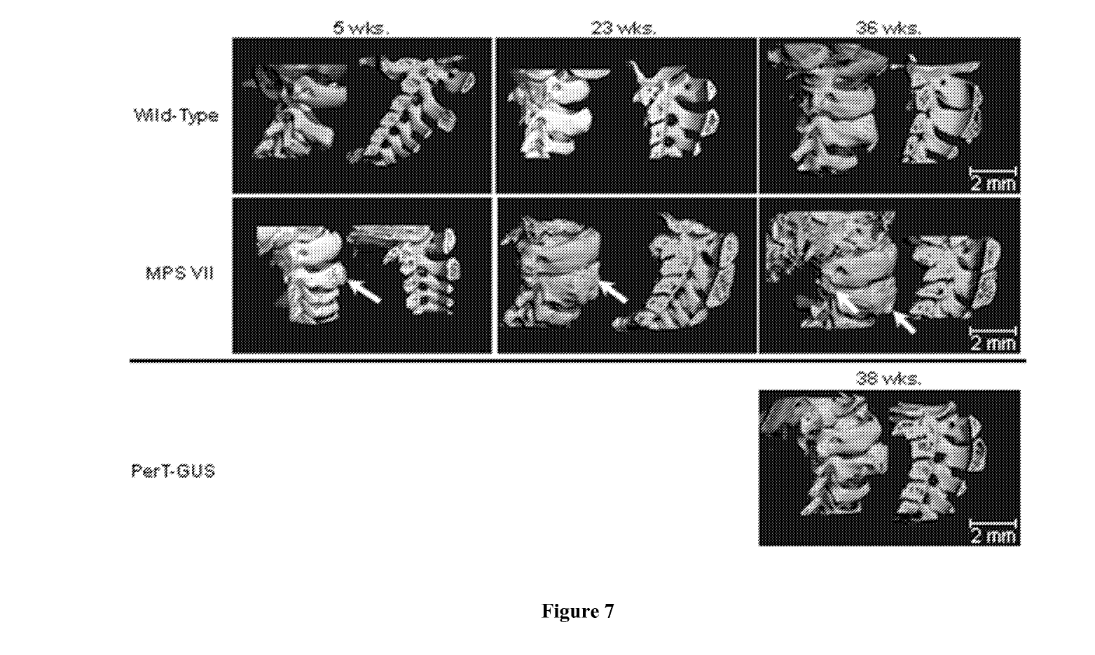

Micro-CT analysis of the bones of the knee joint (FIG. 6) and spine (FIG. 7) in untreated MPS VII mice showed progressive abnormalities with age.

Knee Joints:

At 5 weeks of age, bones in the knee joints of untreated MPS VII mice had modest changes from those of wild-type: 1) less ossified bone, and 2) reduced amounts of trabecular bone (FIG. 6). By 23 weeks of age, the differences in the knee joints were marked. The cortical bone of the tibia and femur were thickened and abnormal periosteal bone formations were observed on the articular surfaces of the tibia and femur. The abnormalities were even more severe in 36 week-old mice (FIG. 6). Micro-CT scans also allowed BMD of mouse knees to be measured. The mean BMD of WT mice over 10 weeks-old was 496.2±38.8 mgHA/mL (n=14) and the mean BMD for untreated MPS VII mice (n=4) was 568.5±66.5 mgHA/mL, which is significantly elevated compared with WT mice (p<0.05).

Treatment effects: PerT-GUS treated MPS VII mice showed marked improvements of the knee joint when compared with those of untreated MPS VII mice. Thicknesses of cortical bone of the femur and tibia were normalized and there were fewer periosteal bone formations, although the knee joints were still distinguishable from wild-type mouse knee joints (FIG. 6). Treatment reduced BMD to 499.7±34.2 mgHA/mL (n=5; p=0.08, compared with untreated MPS VII mice).

Cervical Spine:

At 5 weeks of age, the vertebrae of untreated MPS VII mice appeared to have less ossified bone than those of the wild-type mice (FIG. 7). By 23 weeks of age, the vertebral arches were abnormally thickened and periosteal bone formation was seen on the transverse processes of the vertebrae. The vertebral bodies were flattened and wider (platyspondyly) than those in wild-type mice. In addition, the enlarged vertebrae encroached on the spinal canal causing spinal canal narrowing. These findings were even more prominent in 36-week-old untreated mice.

Treatment effects: A micro-CT scan (n=1) of the spine of a 38-week-old treated MPS VII mouse showed less abnormal thickening of the bone than in untreated MPS VII mice (n=4), resulting in less spinal canal narrowing. In addition, the vertebral bodies were not abnormally wide like those in untreated MPS VII mice (FIG. 7).

Only one cervical spine from a PerT-GUS long-term therapy mouse was available for micro-CT study due to dissection-related damage to CV1-2 on the other specimens.

### Example 5

Radiographic Analysis

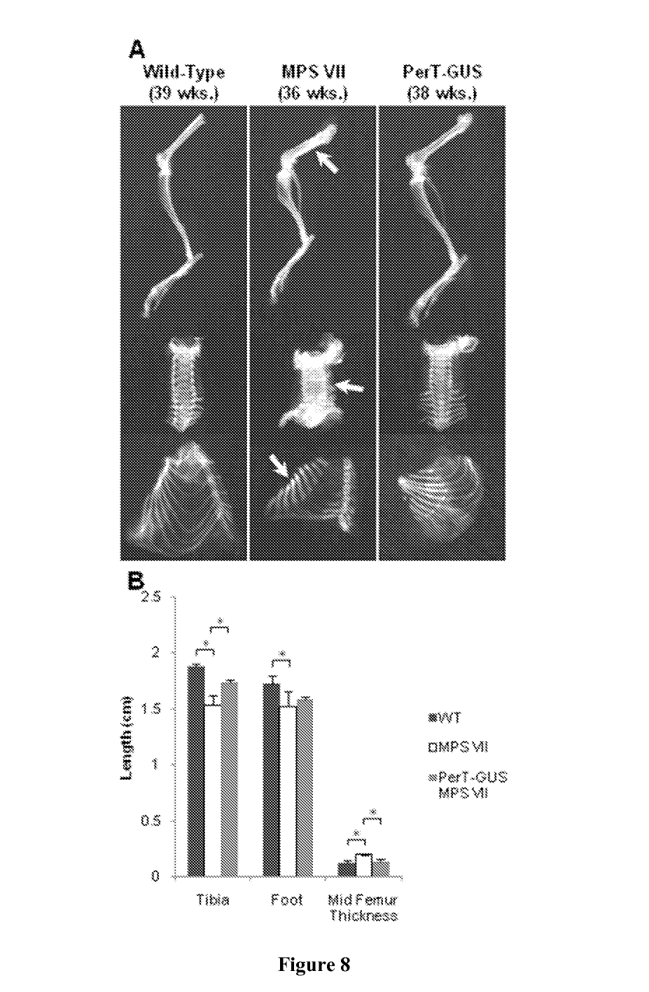

Radiographs comparing the lower extremities of wild-type, untreated MPS VII, and PerT-GUS-treated MPS VII mice are presented in FIG. 8A. The tibias of MPS VII mice older than 10 weeks were shortened (1.54±0.09 mm) when compared with those of wild-type mice (1.88±0.03 mm; p<0.05) (FIG. 8B). The long bones were also broad and sclerotic at 36 weeks of age when compared with those of wild-type mice. The ribcage was narrow with short and thick ribs. The sternal ends of the ribs showed decreased radiodensity on plain radiographs. The cervical vertebrae showed severely increased radiodensity when compared with those in wild-type mice.

Treatment effects: The tibia length of treated MPS VII mice (1.73±0.03 cm) was significantly increased compared with untreated MPS VII mice (1.54±0.09 cm, p<0.05). In addition, the ribs of treated mice were longer and had significantly reduced radiodensity compared with those of untreated mice. The cervical vertebrae of treated MPS VII mice had significantly reduced radiodensity compared with those in untreated mice (FIG. 8A).

### Example 6

Histopathologic Analysis of Knee Joints

Untreated MPS VII Mice

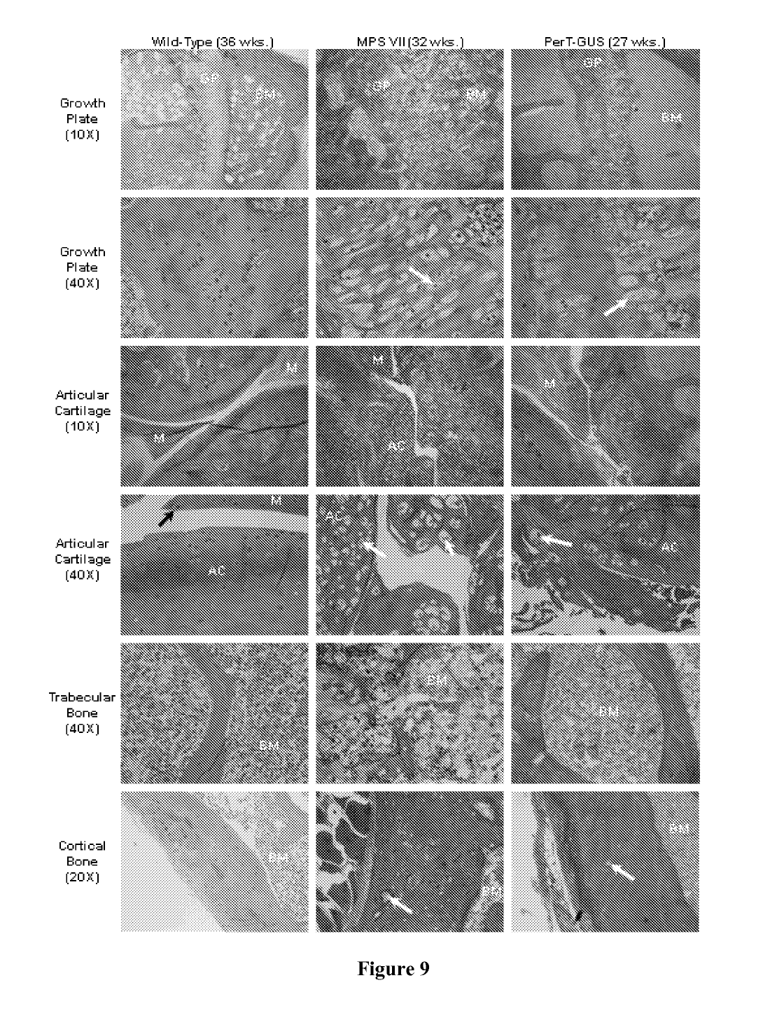

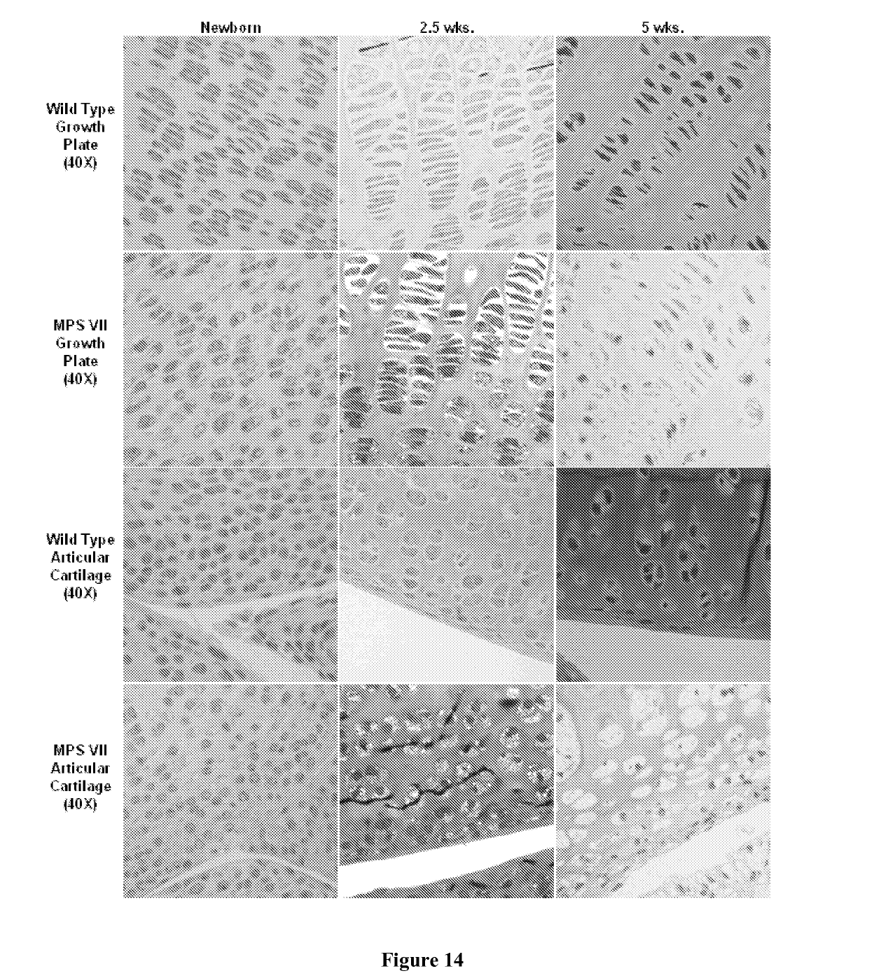

Articular cartilage: The knee joints of affected mice showed noticeable lysosomal storage within the articular cartilage even in the newborn mouse (day 1 or 2). Most articular chondrocytes had vacuoles, although the structure was organized (FIG. 14). Affected mice showed marked lysosomal storage within the articular cartilage by 2.5 weeks of age. The articular cartilage layers (tangential, transitional and radial layers) were abnormally thickened. The chondrocytes were increased in number and ballooned with vacuoles although all three layers were still distinguishable and organized. The 10-week-old affected mice showed abnormal proliferation of the meniscal fibro-cartilage with ballooned vacuolated cells. The articular cartilage layers were slightly irregular and hypercellular, and chondrocytes were enlarged and vacuolated. The three layers were thinner compared with those seen at 2.5 weeks, and their structure was disorganized. The articular cartilage layers at 32 weeks of age showed more disorganization with almost complete loss of the normal arrangement of cells (FIG. 9). The surface of articular cartilage was irregular, and few chondrocytes in the tangential layer were observed. The transitional and radial layers showed hypercellularity compared with those in the age-matched wild-type mice. There were articular-meniscal-synovial fusions with marked abnormal proliferation of articular and meniscal cartilage, with thickened and vacuolated cells in the meniscus and synovium. The synovial space was markedly diminished. All articular cartilage cells showed marked distention, producing a thicker layer. The cells in the periosteum also had marked vacuolor distension.

Growth plate: The growth plate region in 1- or 2-day-old MPS VII mice had ballooned vacuolated chondrocytes in resting and proliferative zones. By 2.5 weeks of age, the growth plate was thickened but showed normal resting and proliferative zonal organization (FIG. 14). The cells were swollen with increased fibrillary or vacuolar contents, which were especially prominent in the resting zone. The hypertrophic zone, although hypercellular, showed disorganization with a distorted arrangement of cells. The primary calcification zone was also increased in size. The longitudinal arrangement of the primary trabeculae was abnormal with the trabeculae increased in number and thickness and contained a marked increase of cartilage. Osteoblasts appeared to be increased in number, especially in the proximal intertrabecular spaces, and contained numerous vacuoles.

At 10 weeks of age, the growth plates were thicker and their boundaries became irregular. The column structure through all layers of the growth plate was disorganized. The chondrocytes were ballooned with vacuoles. The osteoblasts surrounding diaphyseal bone trabeculae and the cells lining bone marrow sinusoids contained a large amount of clear cytoplasmic vacuoles (data not shown).

At 32 weeks of age, the column structure through all layers of the growth plate was markedly disorganized and all chondrocytes were prominently ballooned with vacuoles (FIG. 9). The growth plates had a marked decrease in the number of cells in the proliferating zone. The storage was marked, with lysosomal distention in osteoblasts lining the cortical and trabecular bone and in the sinus-lining cells in the bone marrow. The light microscopic views revealed a loss of the parallel order of the bone matrix with loss of the concentric arrangement of lamellae or haversian system formation. The cortex was markedly thickened in affected mice. The osteocytes showed clearly increased cytoplasmic volumes filled with vacuoles.

### Example 7

PerT-GUS Treated MPS VII Mice:

PerT-GUS treatment from birth to older than 6 months provided substantial improvement in bone pathology. The articular cartilage region showed reduced cellularity and improvement in irregular articular surfaces, although reduction of storage materials in chondrocytes was limited at all cartilage layers. Marked improvement was observed in the abnormal proliferation of articular and meniscal cartilage, leading to reduced articular-meniscal-synovial fusion (FIG. 9). Ligaments and connective tissues surrounding the articular cartilage in treated mice had fewer storage vesicles.

The growth plate region in treated mice showed the following: 1) improvement of architecture by reduction of thickened cartilage layer and irregular surface, and 2) reduced cell area in the proliferative zone, although vacuolated chondrocytes with lysosomal distension remained obvious (FIG. 9). Treated mice had reduced storage materials in bone marrow and restoration of bone architecture. The amount of lysosomal storage vesicles in osteoblasts was markedly reduced. The sinus lining cells in bone marrow and bone marrow cells showed complete clearance of storage vesicles. The osteocytes within the bone had substantially reduced storage material with recovery of cortical bone architecture. These pathological improvements correlated with marked improvements shown on X-ray images.

### Example 8

Quantitative Analysis of Histopatholoqy

Quantitative analysis of the histopathology of wild-type and untreated and treated MPS VII mouse knees was carried out using the same methods described for the comparison of GUS and PerT-GUS treated mice. These measurements supported our histopathological observations. Untreated MPS VII mice (n=4) had thicker growth plates (p<0.005) and articular cartilage (p<0.02) compared with those in wild-type mice (n=5). PerT-GUS treatment (n=5) reduced the thickness of both the growth plate (p=0.14) and articular cartilage (p=0.24) compared with those in the untreated MPS VII mice (FIG. 10A).

The articular cartilage cellularity in untreated MPS VII mice was increased (p<0.001) compared with that in wild-type mice and was significantly reduced by PerT-GUS treatment (p<0.03; FIG. 10B). By contrast, the cellularity in the proliferative zone of growth plate was not different statistically between wild-type, untreated MPS VII, and PerT-GUS-treated mice (FIG. 10C). Cell sizes in the growth plate and articular cartilage were greatly increased in untreated MPS VII mice compared with wild-type mice (growth plate, p<0.001; articular cartilage, p<0.001). PerT-GUS treatment caused a reduction in cell size at the growth plate (p<0.001) and articular cartilage (p<0.03; FIG. 10D). The number of cells/column in the growth plate of MPS VII mice was decreased when compared with wild-type mice (p<0.02) and there was no difference when compared with PerT-GUS treatment (p=0.19; FIG. 10E). The perimeter-to-length ratio of the growth plate in MPS VII mice was elevated when compared with wild-type mice (p<0.001), showing the irregular morphology of the growth plate in untreated MPS VII mice. The perimeter to length ratio was reduced and approached normal in the PerT-GUS-treated mice (p<0.001; FIG. 10F).

All publications and patents cited in this specification are hereby incorporated by reference in their entirety. The discussion of the references herein is intended merely to summarize the assertions made by the authors and no admission is made that any reference constitutes prior art. Applicants reserve the right to challenge the accuracy and pertinence of the cited references.

### REFERENCES

[1] E. F. Neufeld, J. Muenzer, The mucopolysaccharidoses, in: C. R. Scriver, A. I. Beaudet, W. S. Sly, D. Valle (Eds.), Metabolic and Molecular Basis of Inherited Disease, McGraw-Hill, New York, 2001, pp. 3421-3452.
[2] S. Tomatsu, A. M. Montaño, V. C. Dung, J. H. Grubb, W. S. Sly, Mutations and polymorphisms in GUSB gene in mucopolysaccharidosis VII (Sly Syndrome), Hum. Mutat. 30 (2009) 511-519.
[3] C. Vogler, E. H. Birkenmeier, W. S. Sly, B. Levy, C. Pegors, J. W. Kyle, W. G. Beamer, A murine model of mucopolysaccharidosis VII. Gross and microscopic findings in beta-glucuronidase-deficient mice, Am. J. Pathol. 136 (1990) 207-217.
[4] W. S. Sly, C. Vogler, J. H. Grubb, M. Zhou, J. Jiang, X. Y. Zhou, S. Tomatsu, Y. Bi, E. M. Snella, Active site mutant transgene confers tolerance to human beta-glucuronidase without affecting the phenotype of MPS VII mice, Proc. Natl. Acad. Sci. U.S.A. 98 (2001) 2205-2210.
[5] N. W. Barton, F. S. Furbish, G. J. Murray, M. Garfield, R. O. Brady, Therapeutic response to intravenous infusions of glucocerebrosidase in a patient with Gaucher disease, Proc. Natl. Acad. Sci. U.S.A. 87 (1990) 1913-1916.
[6] L. P. Winkel, J. M. Van den Hout, J. H. Kamphoven, J. A. Disseldorp, M. Remmerswaal, W. F. Arts, M. C Loonen, A. G. Vulto, P. A. Van Doorn, G. De Jong, W. Hop, G. P. Smit, S. K. Shapira, M. A. Boer, O. P. van Diggelen, A. J. Reuser, A. T. Van der Ploeg, Enzyme replacement therapy in late-onset Pompe's disease: a three-year follow-up, Ann. Neurol. 55 (2004) 495-502.
[7] M. Rohrbach, J. T. Clarke, Treatment of lysosomal storage disorders: progress with enzyme replacement therapy, Drugs 67 (2007) 2697-2716.
[8] R. Lachmann, Treatments for lysosomal storage disorders, Biochem. Soc. Trans. 38 (2010) 1465-1468.
[9] C. Vogler, M. S. Sands, B. Levy, N. Galvin, E.H. Birkenmeier, W. S. Sly, Enzyme replacement with recombinant beta-glucuronidase in murine mucopolysaccharidosis type VII: impact of therapy during the first six weeks of life on subsequent lysosomal storage, growth, and survival, Pediatr. Res. 39 (1996) 1050-1054.
[10] C. Vogler, M. S. Sands, N. Galvin, B. Levy, C. Thorpe, J. Barker, W. S. Sly, Murine mucopolysaccharidosis type VII: the impact of therapies on the clinical course and pathology in a murine model of lysosomal storage disease, J. Inherit. Metab. Dis. 21 (1998) 575-586.
[11] C. Vogler, B. Levy, J. H. Grubb, N. Galvin, Y. Tan, E. Kakkis, N. Pavloff, W. S. Sly, Overcoming the blood-brain barrier with high-dose enzyme replacement therapy in murine mucopolysaccharidosis VII, Proc. Natl. Acad. Sci. U.S.A. 102 (2005) 14777-14782.
[12] U. Dunder, V. Kaartinen, P. Valtonen, E. Vaananen, V. M. Kosma, N. Heisterkamp, J. Groffen, I. Mononen, Enzyme replacement therapy in a mouse model of aspartyl-glycosaminuria, FASEB J. 14 (2000) 361-367.
[13] D. P. Roces, R. Lüllmann-Rauch, J. Peng, C. Balducci, C. Andersson, O. Tollersrud, J. Fogh, A. Orlacchio, T. Beccari, P. Saftig, K. von Figura, Efficacy of enzyme replacement therapy in alpha-mannosidosis mice: a preclinical animal study, Hum. Mol. Genet. 13 (2004) 1979-1988.
[14] U. Matzner, E. Herbst, K. K. Hedayati, R. Lüllmann-Rauch, C. Wessig, S. Schroder, C. Eistrup, C. Moller, J. Fogh, V. Gieselmann, Enzyme replacement improves nervous system pathology and function in a mouse model for metachromatic leukodystrophy, Hum. Mol. Genet. 14 (2005) 1139-1152.
[15] J. H. Grubb, C. Vogler, B. Levy, N. Galvin, Y. Tan, W. S. Sly, Chemically modified beta-glucuronidase crosses blood-brain barrier and clears neuronal storage in murine mucopolysaccharidosis VII, Proc. Natl. Acad. Sci. U.S.A. 105 (2008) 2616-2621.
[16] M. S. Sands, C. Vogler, J. W. Kyle, J. H. Grubb, B. Levy, N. Galvin, W. S. Sly, E. H. Birkenmeier, Enzyme replacement therapy for murine mucopolysaccharidosis type VII, J. Clin. Invest. 93 (1994) 2324-2331.
[17] E. H. Birkenmeier, M. T. Davisson, W. G. Beamer, R. E. Ganschow, C. A. Vogler, B. Gwynn, K. A. Lyford, L. M. Maltais, C. J. Wawrzyniak, Murine mucopolysaccharidosis type VII. Characterization of a mouse with beta-glucuronidase deficiency, J. Clin. Invest. 83 (1989) 1258-1266.
[18] J. H. Grubb, C. Vogler, Y. Tan, G. N. Shah, A. F. MacRae, W. S. Sly, Infused Fc-tagged beta-glucuronidase crosses the placenta and produces clearance of storage in utero in mucopolysaccharidosis VII mice. Proc Natl. Acad. Sci. U.S.A. 105 (2008) 8375-8380.
[19] A. M. Montano, H. Oikawa, S. Tomatsu, T. Nishioka, C. Vogler, M. A. Gutierrez, T. Oguma, Y. Tan, J. H. Grubb, V. C. Dung, A. Ohashi, K. Miyamoto, T. Orii, Y. Yoneda, W. S. Sly, Acidic amino acid tag enhances response to enzyme replacement in mucopolysaccharidosis type VII mice, Mol. Genet. Metab. 94 (2008) 178-189.
[20] J. H. Grubb, C. Vogler, W. S. Sly, New strategies for enzyme replacement therapy for lysosomal storage diseases, Rejuvenation Res. 13 (2010) 229-236.
[21] Nazarian A, Snyder B D, Zurakowski D, Muller R. (2008). Quantitative micro-computed tomotraphy: A non-invasive method to assess equivalent bone mineral density. Bone. 43: 302-311)
[22] I. Visigalli, S. Delai, L. S. Politi, C. Di Domenico, F. Cerri, E. Mrak, R. D'Isa, D. Ungaro, M. Stok, F. Sanvito, E. Mariani, L. Staszewsky, C. Godi, I. Russo, F. Cecere, U. Del Carro, A. Rubinacci, Brambilla, A. Quattrini, P. Di Natale, K. P. Ponder, L. Naldini, A. Biffi, Gene therapy augments the efficacy of hematopoietic cell transplantation and fully corrects mucopolysaccharidosis type I phenotype in the mouse model, Blood 116 (2010) 5130-5139.
[23] C. Vogler, B. Levy, N. Galvin, M. Lessard, B. Soper, J. Barker, Early onset of lysosomal storage disease in a murine model of mucopolysaccharidosis type VII: undegraded substrate accumulates in many tissues in the fetus and very young MPS VII mouse, Pediatr. Dev. Pathol. 8 (2005) 453-462.
[24] S. Tomatsu, A. M. Montaño, A. Ohashi, M. A. Gutierrez, H. Oikawa, T. Oguma, V. C. Dung, T. Nishioka, T. Orii, W. S. Sly, Enzyme replacement therapy in a murine model of Morquio A syndrome, Hum. Mol. Genet. 17 (2008) 815-824
[25] A. R. Garcia, J. Pan, J. C. Lamsa, J. Muenzer, The characterization of a murine model of mucopolysaccharidosis II (Hunter syndrome), J. Inherit. Metab. Dis. 30 (2007) 924-934.
[26] C. M. Simonaro, M. D'Angelo, X. He, E. Eliyahu, N. Shtraizent, M. E. Haskins, E. H. Schuchman. Mechanism of Glycosaminoglycan-Mediated Bone and Joint Disease, Am. J. Pathol. 172 (2008) 112-122.
[27] J. A. Metcalf, Y. Zhang, M. J. Hilton, F. Long, K. P. Ponder, Mechanism of shortened bones in mucopolysaccharidosis VII, Mol. Genet. Metab. 97 (2009) 202-211.
[28] S. Tomatsu, A. M. Montaño, V. C. Dung, A. Ohashi, H. Oikawa, T. Oguma, T. Orii, L. Barrera, W. S. Sly, Enhancement of drug delivery: enzyme-replacement therapy for murine Morquio A syndrome, Mol. Ther. 18 (2010) 1094-1102.
[29] J. L. Milián, S. Narisawa, I. Lemire, T. P. Loisel, G. Boileau, P. Leonard, S. Gramatikova, R. Terkeltaub, N. P. Camacho, M. D. McKee, P. Crine, M. P. Whyte, Enzyme replacement therapy for murine hypophosphatasia, J. Bone Miner. Res. 23 (2008) 777-787.
[30] T. Nishioka, S. Tomatsu, M. A. Gutierrez, K. Miyamoto, G. G. Trandafirescu, P. L. Lopez, J. H. Grubb, R. Kanai, H. Kobayashi, S. Yamaguchi, G. S. Gottesman, R. Cahill, A. Noguchi, W. S. Sly, Enhancement of drug delivery to bone: characterization of human tissue-nonspecific alkaline phosphatase tagged with an acidic oligopeptide, Mol. Genet. Metab. 88 (2006) 244-255.
[31] C. M. Simonaro, Y. Ge, E. Eliyahu, X. He, K. J. Jepsen, E. H. Schuchman. Involvement of the Toll-like receptor 4 pathway and use of TNF-α antagonists for treatment of the mucopolysaccharidoses, PNAS. 107 (2010) 222-227.
[32]. Banks W A (2004) Are the extracellular [correction of extracelluar] pathways a conduit for the delivery of therapeutics to the brain? Curr Pharm Des 10: 1365-1370.
[33] Chinese Hamster Ovary Cell Line American Type Culture Collection, ATCC CRL 9618.
[34] Ulmasov B, et al. (2000) Purification and kinetic analysis of recombinant CA XII, a membrane carbonic anhydrase over expressed in certain cancers. Proc Natl Acad Sci USA 97: 14212-14217.
[35] Hickman S, Shapiro L J, Neufeld E F (1974) A recognition marker required for uptake of a lysosomal enzyme by cultured fibroblasts. Biochem Biophys Res Commun 57:55-61.
[36] Achord D T, Brot F E, Bell C E, Sly W S (1978) Human p-glucuronidase: In vivo clearance and in vitro uptake by a glycoprotein recognition system on reticuloendothelial cells. Cell 15:269-278.

What is claimed is:

1. A method of administering enzyme replacement therapy to a subject with an lysosomal enzyme deficiency, the method comprising:
a) modifying a lysosomal enzyme determined to be deficient in the subject with sodium meta-periodate treatment followed by treatment with sodium borohydride, wherein clearance from blood is prolonged, and enzyme activity is retained; and
b) administering an effective amount of the modified lysosomal enzyme by intraperitoneal injection.

2. The method of claim 1 wherein the lysosomal enzyme deficiency and deficient enzyme is selected from the group consisting of: Morquio syndrome, deficient in N-acetylgalactosamine-6-sulfatase; Hurler syndrome, deficient in Iduronidase; Hunter syndrome, deficient in Iduronate-2-sulfatase; Sanfilippo syndrome, deficient in Alpha-N-acetylglucosaminidase; Gaucher's disease, deficient in beta-glucosidase; Fabry disease, deficient in alpha-galactosidase; Hurler syndrome, deficient in α-L-iduronidase; Maroteaux-Lamy syndrome, deficient in N-acetylgalactosamine 4-sulfatase; and Pompe disease deficient in acid alpha-glucosidase.

3. The method of claim 1 wherein the lysosomal enzyme deficiency is type VII mucopolysaccharidoses and the deficient lysosomal enzyme is β-glucuronidase

4. The method of claim 1 wherein the intraperitoneal injection or infusions is by way of an intraperitoneal injection port.

5. A method of treating a type VII mucopolysaccharidoses in a subject in need, the method comprising:
a) modifying an isolated β-glucuronidase with sodium meta-periodate treatment followed by treatment with sodium borohydride, wherein clearance from blood is prolonged and enzyme activity is retained; and
b) administering to the subject an effective amount of the modified β-glucuronidase by intraperitoneal injection for an effective period of time.

6. The method of claim 5, wherein an effective amount is about 2 mg per kilogram of the subject to be treated.

7. The method of claim 5, wherein the effective period of time is about 57 weeks.

8. The method of claim 5, wherein the effective period comprises administration on weekly basis for about 6 weeks followed by administration about every 2 weeks for about 51 weeks.

9. The method of claim 5, wherein the effective period begins about 5 weeks after birth.

10. The method of claim 5, wherein the effective period begins about 5 weeks after birth and continues for about 12 weeks.

11. The method of claim 5, wherein the subject sufferers from a bone lesions and bone mineral density is reduced compared to a non-treated subject suffering from a type VII mucopolysaccharidoses related bone lesion.

12. A method of treating a type VII mucopolysaccharidoses related bone lesion in a subject in need, the method comprising:
a) modifying an isolated β-glucuronidase with sodium meta-periodate treatment followed by treatment with sodium borohydride, wherein clearance from blood is prolonged and enzyme activity is retained; and
b) administering to the subject an effective amount of the modified β-glucuronidase for an effective period of time, beginning about 5 weeks after birth.

13. The method of claim 12, wherein an effective amount is about 2 mg per kilogram of the subject to be treated.

14. The method of claim 12, wherein administration is by way of intravenous injection.

15. The method of claim 12, wherein administration is by way of intraperitoneal injection.

16. The method of claim 12, wherein the effective period beginning about 5 weeks after birth, continues for about 12 weeks.

17. The method of claim 12, wherein the subject sufferers from a bone lesions and bone mineral density is reduced compared to a non-treated subject suffering from a type VII mucopolysaccharidoses related bone lesion.
